(19) United States
(12) Patent Application Publication
(10) Pub. No.: US 2013/0338607 A1
(43) Pub. Date: Dec. 19, 2013

(54) Anti-Flashback Needle Adaptor

(71) Applicant: Philip Alexander Paspa, Hickory, NC (US)

(72) Inventor: Philip Alexander Paspa, Hickory, NC (US)

(21) Appl. No.: 13/918,072

(22) Filed: Jun. 14, 2013

Related U.S. Application Data
(60) Provisional application No. 61/689,905, filed on Jun. 15, 2012.

(51) Int. Cl. A61M 39/24; A61B 17/34

(52) U.S. Cl. CPC A61M 39/24; A61B 17/3498
USPC 604/246; 604/533

(57) ABSTRACT

An anti-flashback needle adapter inhibits the uncontrolled jetting of blood upon the insertion of an arterial access needle into a blood vessel. An inner wall defines at least one inner port and an outer wall defines at least one outer port in fluid communication with the inner port. As blood enters the device from the arterial access needle, it is diverted first through the inner port and then through the outer port, which diversion has a baffle effect and dissipates the energy of the blood flow. This prevents the jetting of blood and potential contamination of nearby people and objects. A funnel member allows for the insertion of a guide wire into the arterial access needle while maintaining the blood flow dissipation properties of the device.

### CROSS-REFERENCE TO PRIORITY APPLICATION

This application hereby claims the benefit of pending U.S. Provisional Patent Application No. 61/689,905 for an “Anti-Flashback Needle Adapter” (filed Jun. 15, 2012 at the United States Patent and Trademark Office), which is hereby incorporated by reference in its entirety.

### FIELD OF THE INVENTION

The disclosure relates generally to medical equipment, and, more specifically, to anti-flashback needle adapters.

### BACKGROUND OF THE INVENTION

During the course of medical procedures where percutaneous vascular access is contemplated, an arterial access needle is used. When a medical provider desires to position an object such as a stent into a blood vessel (e.g., vein, artery), the Seldinger technique may be employed. Using this technique, the medical provider inserts a guide wire (e.g., Seldinger wire) through the bore of the arterial access needle into the blood vessel. The guide wire may then be used to introduce the stent or other object into the blood vessel.

During insertion of the arterial access needle into the patient, the medical provider must ensure that the arterial access needle is in the blood vessel or there will not be proper placement of the guide wire. Typically, the medical provider confirms that the arterial access needle is in the proper position by observing the flashback (e.g., flash, backflow), which is the flow of blood out of the patient through the arterial flashback needle. In the case of venous blood flow, the amount of flashback that is obtained from the arterial access needle is minimal as there is no pressure head. When an arterial access needle is placed into an artery, however, the pressure head is much greater, and the blood flows out of the patient with much greater force. Because this arterial flashback can be so forceful, it can result in a blood stream jetting a great distance from the arterial access needle. This jetting or pulsing effect increases the likelihood of blood hitting the medical professional or assistants, or of blood contaminating nearby surfaces or fomites.

There exists a need, therefore, for an adapter for an arterial access needle that would reduce the likelihood of blood contamination of surrounding people and objects from flashback during the introduction of the arterial access needle into a blood vessel, particularly into an artery.

### SUMMARY OF THE INVENTION

The disclosure relates to an anti-flashback needle adapter. In one aspect, the disclosure embraces an anti-flashback needle adapter for an arterial access needle. The anti-flashback needle adapter has a proximal end portion and a distal end portion. The anti-flashback needle adapter also has an inner wall defining at least one inner offset port, and an outer wall defining at least one outer offset port. The at least one inner offset port is in fluid communication with the at least one outer offset port.

In an alternative exemplary embodiment of the anti-flashback needle adapter according to the present disclosure, the at least one inner offset port comprises at least two inner offset ports positioned substantially adjacent to the proximal end portion of the anti-flashback needle adapter and at least two inner offset ports positioned substantially adjacent to the distal end portion of the anti-flashback needle adapter.

In an alternative exemplary embodiment of the anti-flashback needle adapter according to the present disclosure, the at least one outer offset port comprises at least four outer offset ports positioned substantially adjacent to the distal end portion of the anti-flashback needle adapter.

The anti-flashback needle adapter may also have a funnel member for guiding a guide wire through the anti-flashback needle adapter into the arterial access needle. The funnel member defines a funnel opening through which the guide wire may be inserted. The funnel member may be fitted with a funnel valve capable of inhibiting the flow of blood through the funnel opening toward the proximal end portion of the anti-flashback needle adapter and capable of allowing a guide wire to be inserted through the funnel opening from the proximal end portion of the anti-flashback needle adapter. The anti-flashback needle adapter may also have a needle connector for connecting an arterial access needle to the anti-flashback needle adapter.

In another aspect, the disclosure embraces an arterial access needle unit. The arterial access needle unit includes an arterial access unit connected to an anti-flashback needle adapter having a proximal end portion and a distal end portion. The anti-flashback needle adapter has an inner wall defining at least one inner offset port, and an outer wall defining at least one outer offset port. The at least one inner offset port is in fluid communication with the at least one outer offset port.

The foregoing, as well as other objectives and advantages of the disclosure, and the manner in which the same are accomplished, are further specified within the following detailed description and its accompanying drawings.

### BRIEF DESCRIPTION OF THE DRAWINGS

FIG. 1 is a side cross-sectional view of an exemplary anti-flashback needle adapter according to the present disclosure.

FIG. 2 is a bottom cross-sectional view of an exemplary anti-flashback needle adapter according to the present disclosure.

FIG. 3 is a side cross-sectional view of an exemplary arterial access needle unit according to the present disclosure.

FIG. 4 is a bottom perspective view of an exemplary anti-flashback needle adapter according to the present disclosure.

### DETAILED DESCRIPTION

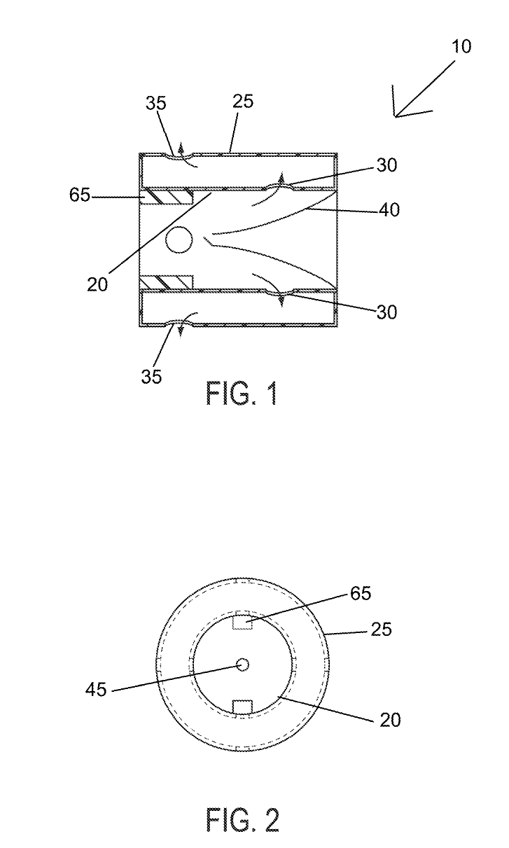

The disclosure relates to an anti-flashback needle adapter. In one aspect, the disclosure embraces an anti-flashback needle adapter for an arterial access needle. Reference will now be made to FIGS. 1 through 4. In this disclosure, a convention is followed where the term “proximal” refers to the portion of the device closest to the medical provider (e.g., practitioner, user), and the term distal refers to the portion of the device nearest the patient (e.g., farthest from the medical provider). The anti-flashback needle adapter 10 has a proximal end portion and a distal end portion. The anti-flashback needle adapter also has an inner wall 20 and an outer wall 25. The inner wall 20 defines at least one inner offset port. The outer wall 25 defines at least one outer offset port.

Typically, the inner wall 20 defines at least two inner offset ports 30 positioned substantially adjacent to the proximal end portion of the anti-flashback needle adapter 10 and at least two inner offset ports 30 positioned substantially adjacent to the distal end portion of the anti-flashback needle adapter 10. Typically, outer wall 25 defines at least four outer offset ports 35. The inner offset ports 30 are in fluid communication with the outer offset ports 35 such that blood may flow through the inner offset ports 30 to the outer offset ports 35.

As shown in FIG. 1, when blood enters through the distal end portion of the arterial access needle 10, the blood flows out of the arterial access needle 10 first through the inner offset ports 30 and then through the outer offset ports 35. In FIG. 1 the curved arrows indicate the direction of the flow of blood first through the inner offset ports and then out through the outer offset ports. By placing the inner offset ports 30 substantially adjacent to the proximal end portion of the anti-flashback needle adapter 10 and the outer offset ports 35 substantially adjacent to the distal end portion of the anti-flashback needle adapter 10, the blood is forced to change direction as it flows out of the anti-flashback needle adapter 10. In this way, the anti-flashback needle adapter 10 serves as a baffle chamber, reducing the force of the blood flow. As a result of this baffling effect, the blood will flow out of the anti-flashback needle adapter 10 through the outer offset ports 35 without substantial jetting or pulsing. Instead, the blood will flow out relatively near the anti-flashback needle adapter 10, allowing gravity to disperse the blood in a predictable fashion as opposed to an unpredictable and undesirable jetting effect that can lead to contamination of surrounding people and areas.

A funnel member 40 is positioned at the proximal end portion of the anti-flashback needle adapter 10. The funnel member 10 may be integral to or fixably attached to the anti-flashback needle adapter 10. The funnel member defines a funnel opening 45. A funnel valve 50 may be movably attached to the funnel member 40. The funnel valve 50 is positioned such that when blood enters the anti flashback needle adapter 10 from an arterial access needle 60, the blood will be blocked from passing through the funnel opening 45. This blocking results in the blood being diverted out through the inner offset ports 30. The funnel valve 50 is configured to allow a guide wire to be inserted through the funnel opening 45 from the proximal end portion of the anti-flashback needle adapter 10. This allows the medical practitioner to pass a guide wire through the anti-flashback needle adapter 10 and into the arterial access needle 60, and subsequently into the patient's blood vessel. In this way, the anti-flashback needle adapter 10 inhibits the uncontrolled spurting of blood from the arterial access needle 60 while still allowing for the insertion of a guide wire into the arterial access needle 60.

In an alternative embodiment, the funnel opening 45 may not be covered by a funnel valve 50. Instead, the funnel opening 45 may be positioned in a way that inhibits the flow of blood through the funnel opening 45 as the blood enters the distal end of the anti-flashback needle adapter 10. For example, the funnel opening 45 may be positioned substantially adjacent to the inner wall 20. In this configuration, when blood pulses out of the arterial access needle 60, it will strike the bottom portion of the funnel member 40, thereby dissipating much of the energy of the blood flow. The blood flow will then pass through the inner offset ports 30 in the usual way. The funnel opening 45 in this positioning will still permit the insertion of a guide wire, which insertion could be facilitated further by a similar funneling configuration on the proximal end of the arterial access needle.

The anti-flashback needle adapter 10 may have a needle connector 65 for connecting to an arterial access needle 60. Typically, the needle connector 65 will be fixably attached or integral to the distal end portion of the anti-flashback needle adapter 10, thereby allowing for connection of the distal end portion of the anti-flashback needle adapter 10 to the proximal end portion of the arterial access needle 60. Typically, the needle connector is a Luer-lock connector allowing the anti-flashback needle adapter 10 to be removably attached to the arterial access needle 60.

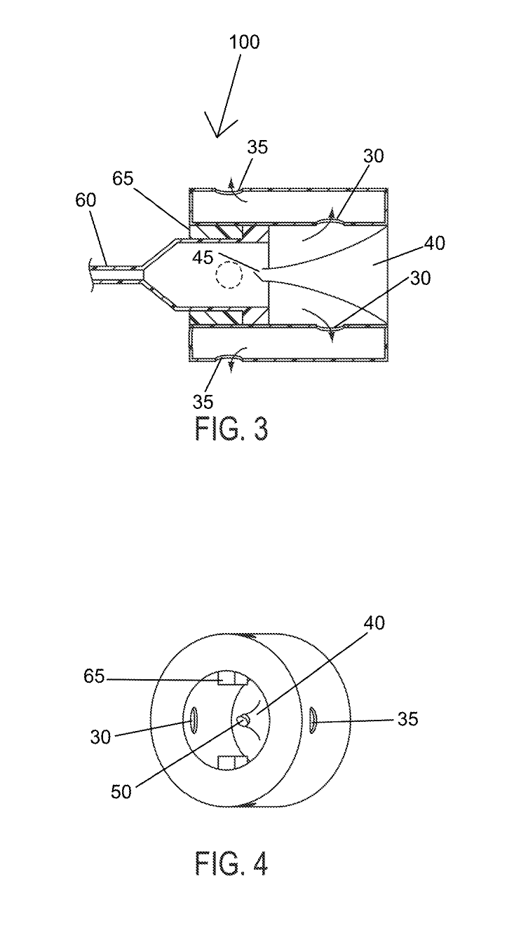

In another embodiment, the disclosure embraces an arterial access needle unit 100. The arterial access needle unit 100 includes an arterial access needle 60 and an anti-flashback needle adapter 10 according to the present disclosure. The arterial access needle 60 may be fixably attached or removably attached to the anti-flashback needle adapter 10. Typically, the arterial access needle unit 100 includes an arterial access needle 10 removably attached to an anti-flashback needle adapter 10 via a Luer-lock connection as shown in FIG. 3.

In the specification and figures, typical embodiments of the invention have been disclosed. The present invention is not limited to such exemplary embodiments. Unless otherwise noted, specific terms have been used in a generic and descriptive sense and not for purposes of limitation.

1. An anti-flashback needle adapter for an arterial access needle, the anti-flashback needle adapter having a proximal end portion and a distal end portion, comprising:
an inner wall defining at least one inner offset port; and
an outer wall defining at least one outer offset port.

2. The anti-flashback needle adapter of claim 1, wherein the at least one inner offset port is in fluid communication with the at least one outer offset port.

3. The anti-flashback needle adapter of claim 2, wherein the at least one inner offset port comprises at least two inner offset ports positioned substantially adjacent to the proximal end portion of the anti-flashback needle adapter and at least two inner offset ports positioned substantially adjacent to the distal end portion of the anti-flashback needle adapter.

4. The anti-flashback needle adapter of claim 2, wherein the at least one outer offset port comprises at least four outer offset ports positioned substantially adjacent to the distal end portion of the anti-flashback needle adapter.

5. The anti-flashback needle adapter of claim 2, comprising a funnel member for guiding a guide wire through the anti-flashback needle adapter into the arterial access needle, the funnel member defining a funnel opening through which the guide wire may be inserted.

6. The anti-flashback needle adapter of claim 5, comprising a funnel valve capable of inhibiting the flow of blood through the funnel opening toward the proximal end portion of the anti-flashback needle adapter and capable of allowing a guide wire to be inserted through the funnel opening from the proximal end portion of the anti-flashback needle adapter.

7. The anti-flashback needle adapter of claim 2, comprising a needle connector for connecting an arterial access needle to the anti-flashback needle adapter.

8. The anti-flashback needle adapter of claim 7, wherein the needle connector comprises a Luer-lock connector.

9. A anti-flashback needle adapter for an arterial access needle, the anti-flashback needle adapter having a proximal end portion and a distal end portion, comprising:
an inner wall defining at least two inner offset ports positioned substantially adjacent to the proximal end portion of the anti-flashback needle adapter and defining at least two inner offset ports positioned substantially adjacent to the distal end portion of the anti-flashback needle adapter;
an outer wall defining at least four outer offset ports positioned substantially adjacent to the distal end portion of the anti-flashback needle adapter;
a funnel member for guiding a guide wire through the anti-flashback needle adapter into the arterial access needle, the funnel member defining a funnel opening through which the guide wire may be inserted; and
a funnel valve capable of inhibiting the flow of blood through the funnel opening toward the proximal end portion of the anti-flashback needle adapter and capable of allowing a guide wire to be inserted through the funnel opening from the proximal end portion of the anti-flashback needle adapter
wherein the inner offset ports are in fluid communication with the outer offset ports.

10. An arterial access needle unit, comprising:
an arterial access needle;
a anti-flashback needle adapter having a proximal end portion and a distal end portion, the anti-flashback needle adapter connected to the arterial access needle, and the anti-flashback needle adapter comprising an inner wall defining at least one inner offset port, and an outer wall defining at least one outer offset port.

11. The arterial access needle unit of claim 10, wherein the at least one inner offset port is in fluid communication with the at least one outer offset port.

12. The arterial access needle unit of claim 11, wherein the at least one inner offset port comprises at least two inner offset ports positioned substantially adjacent to the proximal end portion of the anti-flashback needle adapter and at least two inner offset ports positioned substantially adjacent to the distal end portion of the anti-flashback needle adapter.

13. The arterial access needle unit of claim 11, wherein the at least one outer offset port comprises at least four outer offset ports positioned substantially adjacent to the distal end portion of the anti-flashback needle adapter.

14. The arterial access needle unit of claim 11, comprising a funnel member for guiding a guide wire through the anti-flashback needle adapter into the arterial access needle, the funnel member defining a funnel opening through which the guide wire may be inserted.

15. The arterial access needle unit of claim 14, comprising a funnel valve capable of inhibiting the flow of blood through the funnel opening toward the proximal end portion of the anti-flashback needle adapter and capable of allowing a guide wire to be inserted through the funnel opening from the proximal end portion of the anti-flashback needle adapter and into the arterial access needle.

16. The arterial access needle unit of claim 11, wherein the anti-flashback needle adapter comprises a needle connector for connecting an arterial access needle to the anti-flashback needle adapter.

17. The arterial access needle unit of claim 16, wherein the needle connector comprises a Luer-lock connector.
